(19) United States
(12) Patent Application Publication
(10) Pub. No.: US 2025/0345094 A1
(43) Pub. Date: Nov. 13, 2025

(54) PROGRAMMER FOR USE IN A MOTORIZED SPATIAL FRAME

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Sied W. Janna, Jr., Memphis, TN (US); Johnny R. Mason, Bartlett, TN (US); Darren J. Wilson, Hull (GB); Paul Bell, Vestavia Hills, AL (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/854,117

(22) PCT Filed: Jun. 13, 2023
(86) PCT No.: PCT/US2023/025168

§ 371 Date: Oct. 4, 2024

Related U.S. Application Data
(60) Provisional application No. 63/353,083, filed on Jun. 17, 2022.

(51) Int. Cl. A61B 17/62; A61B 17/00; A61B 17/64; A61B 90/00

(52) U.S. Cl. CPC A61B 17/62; A61B 17/645; A61B 90/08; A61B 2017/00221; A61B 2017/00398; A61B 2017/00477; A61B 2017/00734; A61B 2017/00876; A61B 2090/0808

(57) ABSTRACT

A detachable programmer arranged and configured to be selectively coupled to a motorized strut in an automated and/or motorized spatial frame is disclosed. In some examples, a plurality of detachable programmers are provided, one for each of the plurality of motorized struts in the spatial frame. Thus arranged, each of the plurality of detachable programmers can be configured to supply power and/or control the motorized strut to which it is connected to actuate the motorized strut according to a treatment plan negating, or at least minimizing, the need for any complex, sensitive circuitry housed within the motorized strut. In some examples, the detachable programmer is configured to magnetically couple to the motorized strut via a magnetic or bayonet-style connector.

### CROSS-REFERENCE TO RELATED APPLICATIONS

This is a non-provisional of, and claims the benefit of the filing date of, pending U.S. provisional patent application No. 63/353,083, filed Jun. 17, 2022, entitled “Programmer for Use in a Motorized Spatial Frame,” the entirety of which application is incorporated by reference herein.

### FIELD OF THE DISCLOSURE

The present disclosure relates generally to orthopedic devices, systems, and methods for facilitating fracture alignment such as the treatment of musculoskeletal conditions with a spatial frame, and more particularly to a motorized strut to be used in the spatial frame and a programmer arranged and configured to detachably couple to the motorized strut. In some examples, the programmer is arranged and configured to be selectively attached to, and detached from, the motorized strut via a magnetic or bayonet-style connector. In use, the programmers can be docked onto each of the motorized struts, either individually or sequentially, and used to actuate, control, and/or supply power to the motorized strut according to a treatment plan negating, or at least minimizing, the need for any complex, sensitive circuitry housed within the motorized strut.

### BACKGROUND OF THE DISCLOSURE

People suffer bone fractures each year. In many instances, a person that suffers a bone fracture is required to use a bone alignment device, an external fixation system, etc. such as, for example, a spatial frame, a hexapod, etc. (terms used interchangeably herein without the intent to limit or distinguish) to align two or more bones, bone fragments, bone pieces, etc. (terms used interchangeably herein without the intent to limit or distinguish). Generally speaking, spatial frames allow for polyaxial movement of the coupled bones and are typically used to keep fractured bones stabilized and in alignment during a treatment period.

Generally speaking, the spatial frame includes first and second rings, platforms, frames, bases, etc. (terms used interchangeably herein without the intent to limit or distinguish) intercoupled by a plurality of struts. In use, the struts have adjustable lengths that may be manually adjusted regularly (e.g., daily) in accordance with a prescription or treatment plan (terms used interchangeably herein without the intent to limit or distinguish). As the lengths of the struts are adjusted, the platforms may be brought closer together or moved farther apart. The treatment plan specifies strut length adjustments to be made to each of the struts over time to ensure successful bone alignment.

One known example of a spatial frame is the TAYLOR SPATIAL FRAME® manufactured and sold by Smith Nephew, Inc. The TAYLOR SPATIAL FRAME® is based on the general concept of a Stewart platform. Smith & Nephew, Inc. is the owner of U.S. Pat. Nos. 5,702,389; 5,728,095; 5,891,143; RE40,914, 5,971,984; 6,030,386; and 6,129,727; and U.S. Published Patent Application Nos. 20030191466; 2004/0073211; 2005/0215997; and 2016/0092651 that disclose many concepts of and improvements to the Stewart platform based spatial frame, including methods of use, systems, and devices that enhance use of the spatial frame.

Referring to FIG. 1 one known example of a spatial frame 100 is illustrated. As shown in FIG. 1, the spatial frame 100 may form a hexapod having a circular, metal frame with a first platform 102 and a second platform 104 connected by six adjustable length struts 106 (labeled as struts 106-1 through 106-6 in FIG. 1). Each strut 106 may be independently lengthened or shortened relative to the rest of the frame, thereby allowing for six different axes of movement.

Each strut 106 may include an outer body and an inner body, which may be configured as, or be operatively coupled to, a threaded rod (also referred to as a lead screw). The outer body may be coupled to one of the platforms, such as, the second platform 104 by way of a joint as shown. The inner body may be coupled to the other platform, such as, the first platform 102 by way of a joint as shown. To lengthen or shorten one of struts 106, the outer body and the inner body may be moved or translated relative to one another. For example, the strut 106 may include an adjustment nut wherein rotation of the adjustment nut moves the inner body (e.g., lead screw) relative to the outer body to adjust an overall length of the strut.

In use, the spatial frame 100 may be used to treat a variety of skeletal fractures of a patient. Typically, the spatial frame 100 is positioned around the patient's bone and is used to align two or more bone portions. To do so, a length of each strut 106 may be incrementally adjusted (e.g., shortened or lengthened) in accordance with a treatment plan that specifies adjustments to be made to each strut 106 over time to ensure successful bone alignment. In many instances, the length of each strut 106 should be adjusted daily to comply with the provided treatment plan. Adjusting the length of each strut 106 adjusts the distance and/or position between the first and second platforms 102, 104, and hence the first and second bone portions coupled thereto.

During use, patient's bones are normally adjusted (e.g., lengthened, shortened, etc.) manually, for example, by hand or a wrench at a rate of approximately 1 mm/day, which is then proceeded by a consolidation phase before the spatial frame is removed.

It is theoretically known in the prior art to automate and/or motorize adjustment of a spatial frame by motorizing or otherwise automating strut adjustments. For example, one known motorized strut is the Robotic Hexapod System manufactured by Orthospin Ltd. The Robotic Hexapod System however suffers from a number of disadvantages including being very bulky and having trailing cables, which couple each of the motorized struts to a centralized controller or control unit (terms used interchangeably herein without the intent to limit or distinguish) positioned on one of the platforms.

However, currently commercially available spatial frames are dependent on manual adjustment of each strut. As a result of the requirement for manual adjustments, generally speaking, successful treatment requires patient compliance (e.g., daily manual adjustments to each of the struts) to avoid human error. In routine clinical practice, the treatment plan may require multiple daily adjustments to be made to each of the plurality of struts. For example, a patient may be required to manually adjust one or more of the struts, typically two or more times each day, and often over long periods of time with support from either a family member, a clinician, or both. As such, compliance with the treatment plan may be burdensome, painful, and prone to errors, which may rise as the number of daily adjustments increase.

As a result, the number of adjustments dictated by the treatment plan may be limited. For example, generally speaking, treatment plans often limit the required number of daily adjustments to each of the plurality of struts to four per day. During a normal treatment plan, this may equate to approximately 720 adjustments (e.g., turns) over a one-month treatment span (e.g., 6 struts×4 adjustments per day×30 days). During an extended treatment plan for more severe applications, this may equate to approximately 2,160 adjustments (e.g., turns) over a three-month treatment span (e.g., 6 struts×4 adjustments per day×90 days).

In addition, during the treatment period, the patient may require numerous clinical visits to confirm proper strut adjustments to ensure compliance and avoid incorrect adjustment, which has historically been the leading cause of treatment failure.

Motorized and/or automated spatial frames could provide numerous advantages over manually adjustable struts. In use, electric motors, motor-drive units, and a control unit (e.g., a central control unit) could function to supersede the manual actuation of the strut adjustments. For example, an automated and/or motorized system could eliminate the need for patient compliance and decrease the frequency of post-operative visits for patient supervision given that the spatial frame may only need to be activated at the start of the distraction phase and terminated at the end of the distraction phase without any patient intervention. As a result, the burden of manual adjustment can be overcome by automating and/or motorizing the struts, which in turn, enables a more independent lifestyle during treatment.

In addition, as a programmable multi-purpose device, automated and/or motorized spatial frames allow the implementation of more diverse treatment schedules. For example, automatic and/or motorized distraction could enable a higher distraction frequency and result in smaller excursions per activation. Smaller excursions or adjustments have the potential to result in less damage to the distracted tissues, improving bone regeneration and adaptation of the surrounding soft tissues. That is, spatial frames equipped with motorized and/or automated struts offer the potential to increase the number of daily distraction adjustments by enabling finer (e.g., smaller) adjustments at a controllable rate and frequency of distraction that encourages better quality bone formation. Making finer (e.g., smaller) adjustments during limb lengthening can have significant advantages in terms of reduced soft tissue damage, less pain, and opioid usage and accelerated bone healing. One study has found that the bone fixation index was only 5-6 days/cm when using motorized and/or automated distraction compared to 22-24 days/cm by manual adjustment.

For example, a motorized strut could be programmed to perform anywhere from one adjustment per day to continuous adjustments. Finer adjustments could increase the number of adjustments over a one-month period from approximately 720 adjustments to approximately 3,600 adjustments (e.g., 6 struts×20 adjustments per day×30 days). Alternatively, finer adjustments could increase the number of adjustments over a one-month period to approximately 259,200 adjustments (e.g., 6 struts×1440 adjustments per day×30 days). Over an extended three-month treatment period, this could increase the number of adjustments from approximately 2,160 adjustments to approximately 10,800 adjustments (e.g., 6 struts×20 adjustments per day×90 days). Alternatively, finer adjustments could increase the number of adjustments over a three-month period to approximately 777,600 adjustments (e.g., 6 struts×1440 adjustments per day×90 days).

In use, each motorized strut may include a motor and may be used in a spatial frame such as, for example, spatial frame 100, to move the first and second platforms 102, 104, respectively, to align two or more bone portions. In use, the spatial frame and/or system architecture may be arranged and configured to automatically adjust the motorized struts according to the prescribed treatment plan (e.g., automatically adjust the plurality of motorized struts without patient intervention). Alternatively, the spatial frame and/or system architecture may be arranged and configured to require patient and/or caregiver activation to begin the process of automatically adjusting the motorized struts according to the prescribed treatment plan. For example, the spatial frame may be arranged to intermittently auto-adjust the motorized struts at predetermined times according to the treatment plan. Alternatively, the spatial frame may be arranged to intermittently auto-adjust the motorized struts at select times when convenient and/or selected by the patient. Alternatively, the spatial frame may be arranged and configured to continuously auto-adjust the motorized struts in small discrete increments.

Referring to FIG. 2, one known example of a motorized strut 200 is disclosed. In use, for example, the motorized strut 200 may be coupled to first and second platforms in a spatial frame. For example, the motorized strut 200 may be used in place of the manually adjustable struts 106 shown in FIG. 1. As shown in FIG. 2, the motorized strut 200 may include an outer body 202 operatively coupled with a first joint 204 for coupling to a first platform, an inner body 210 operatively coupled with a second joint 212 for coupling to a second platform, and a drive mechanism, actuator, etc. 220 (used interchangeably herein without the intent to limit or distinguish). In use, actuation of the drive mechanism 220 moves the inner body 210 relative to the outer body 202 to adjust a length of the motorized strut 200.

As illustrated, the drive mechanism 220 may include a motor 222 and a lead screw 224 arranged and configured so that, in use, actuation of the motor 222 rotates the lead screw 224, which moves the inner body 210 relative to the outer body 202 to adjust an overall length of the motorized strut 200. In addition, the drive mechanism 220 may include one or more gears to adjust speed and torque of the motor 222.

In addition, the motorized strut 200 may include any required circuitry. That is, automated or autonomous, motorized spatial frames may incorporate a number of mechanical and electrical components. For example, each motorized strut may include an encoder that senses its rotational positioning and a control circuit that controls the motor speed and direction according to the treatment plan. A motor control circuit may also provide hardware and software protections that prevent any deviation from the treatment plan and alert the patient in the event of a malfunction. In addition, the motorized struts may also house a power supply, a charging circuit, and a wireless communication chip to allow data to be transmitted to and from the strut to an APP or central base station or external computing system.

For example, as illustrated in FIG. 2, the motorized strut 200 may include one or more position sensors to, for example, monitor absolute position or length of the motorized strut 200. In addition, and/or alternatively, the motorized strut 200 may include other sensors for monitoring various biomechanical parameters such as, for example, a force sensor 230 for monitoring stresses and forces, across the bone gap and/or the soft tissues (muscle, apposing cartilage or peripheral sensory nerves), an accelerometer for capturing patient ambulation data (steps, distance, speed and cadence), a gyroscope for measuring the degree of alignment between the bone fragments, and a sensor motor support 232, etc. In addition, and/or alternatively, the motorized strut 200 may include an encoder such as, for example, a rotary encoder for measuring rotation of the motor 222 for accurate positioning and motion control. In addition, and/or alternatively, the motorized strut 200 may include flash memory for storing unique identifiers (e.g., addresses) and for storing current position, biomechanical and ambulatory data, etc.

Additional information on examples of motorized spatial frames can be found in International Patent Application No. PCT/US20/52276, filed on Sep. 23, 2020, published as WO 2021/061816 A1, entitled “Automated Spatial Frame and Automated Struts Used Therewith,” and International Patent Application No. PCT/US23/13011, filed on Feb. 14, 2023, entitled “Detachable Geared-Motor Assembly for Motorizing a Strut in a Spatial Frame,” the entire contents of said application being hereby incorporated in its entirety herein.

However, motorized struts face a number of challenges that need to be overcome. For example, motorized struts are challenging to manufacture. The electronic components housed within the motorized struts are subject to sterilization prior to use, which could adversely affect the performance of the heat-sensitive components including, for example, the control circuitry (e.g., microprocessor) and the power supply (e.g., batteries, coin cells, etc.).

Moreover, in cases where a motorized strut needs to be changed-out or replaced by an alternate motorized strut during the treatment period (e.g., due to a prescription modification or a motor failure), additional complexity to the correction phase of the procedure may be experienced. In addition, currently known motorized spatial frames are cumbersome and bulky in design, especially when considering that a patient has to wear the spatial frame for several weeks if not months. In particular, the presence of a centralized controller and corresponding cables coupling the motorized struts to the centralized controller provide a safety hazard to the patient and limit the patient's mobility.

For example, as previously mentioned, one currently known motorized spatial frame is the Robotic Hexapod System manufactured by Orthospin Ltd. The Robotic Hexapod System is a motorized spatial frame that allows real-time physician follow-up and reduce dependence on patient compliance. In use, the Robotic Hexapod System can automatically and continuously adjust (e.g., lengthen and/or shorten) the struts according to the prescribed treatment plan, without patient involvement. The Robotic Hexapod System utilizes a detachable geared-motor assembly. During use, the detachable geared-motor assemblies can be coupled to custom struts via a first spur gear associated with the motor engaging a second spur gear associated with the lead screw of the strut. In use, rotation of the motor drives rotation of the lead screw via the interaction between the first and second spur gears.

However, the Robotic Hexapod System from Orthospin, Ltd. suffers from several disadvantages. For example, the detachable geared-motor assemblies of the Robotic Hexapod System are powered and controlled by a wired connection to a centralized control unit, which is coupled on top of the circular hexapod fixation platform.

A similar motorized spatial frame was disclosed in “Bone mounted hexapod robot for outpatient distraction osteogenesis” by Wendlandt et al. The motorized spatial frame includes detachable geared-motor assemblies, which are coupled in parallel to six telescopic struts. In use, the detachable geared-motor assemblies are interchangeable with the manual elements thus allowing easy mounting after the operation. The motor engages the manual strut via gears, which allows the lead screw to move in either direction to lengthen or shorten the strut. Furthermore, the motorized spatial frame includes a centralized control unit, which is permanently mounted onto one of the platforms to allow for autonomous adjustments of the struts. The centralized control unit is connected to each of the detachable geared-motor assemblies via a digital two-wire bus USB connection providing power and positional data.

It would be beneficial to provide an automated and/or motorized spatial frame that includes motorized struts having a simplified design and construction. It is with respect to these and other considerations that the present disclosure may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

A motorized spatial frame including a first platform, a second platform, a plurality of motorized struts coupled to the first and second platforms, and one or more detachable programmers is disclosed. In some examples, each of the plurality of motorized struts include an outer body, an inner body, a lead screw coupling the inner body to the outer body, a motor arranged and configured to rotate the lead screw to move the inner body relative to the outer body, and a coupler. That is, each of the plurality of motorized struts include an outer body, a lead screw, and a motor operatively coupled to the lead screw for adjusting a position of the lead screw relative to the outer body to adjust a position of the first platform relative to the second platform.

In some examples, each of the one or more detachable programmers includes a coupler arranged and configured to selectively engage the coupler of the motorized strut so that the detachable programmer can be selectively attached and detached from the motorized strut.

In any preceding or subsequent example, the one or more detachable programmers include a plurality of detachable programmers, one for each of the plurality of motorized struts.

In any preceding or subsequent example, each of the detachable programmers is configured to supply power and to control activation of the motorized strut to which the detachable programmer is attached to, the detachable programmer configured to actuate the motorized strut according to a treatment plan.

In any preceding or subsequent example, the couplers are arranged and configured to mechanically and electrically couple the detachable programmer to the motorized strut.

In any preceding or subsequent example, the coupler of the motorized strut and the coupler of the detachable programmer are arranged and configured as magnetic couplers so that the detachable programmer is magnetically attached to the motorized strut.

In any preceding or subsequent example, the magnetic couplers include a Pogo interface connector or magnetic pogoes including male pins and female connectors in one of a 4-pin, 6-pin, or 8-pin configuration.

In any preceding or subsequent example, each of the detachable programmers include one or more microcontrollers or microprocessors arranged and configured to control the detachable programmer and the motorized strut to which it is coupled.

In any preceding or subsequent example, the one or more microcontrollers or microprocessors are arranged and configured to control operation of the motorized strut including controlling activation of the motor and receiving and updating a treatment plan.

In any preceding or subsequent example, each of the detachable programmers include one or more power supplies arranged and configured to power the detachable programmer and to supply power to the motorized strut to which it is coupled.

In any preceding or subsequent example, each of the detachable programmers include a wireless communication chip arranged and configured to wirelessly communicate with an external computing system to exchange data relating to a treatment plan, to exchange data relating to a status of the motorized strut, to exchange data relating to a health or activity of the patient, or a combination thereof.

In any preceding or subsequent example, each of the detachable programmers include a microprocessor or microcontroller arranged and configured to control operation of the motorized strut coupled thereto, a power supply arranged and configured to power the motorized strut coupled thereto, and a wireless communication chip arranged and configured to transmit and receive data with an external computing system.

In any preceding or subsequent example, each of the detachable programmers is arranged and configured to identify which one of the plurality of motorized struts it is to be coupled to.

In any preceding or subsequent example, each of the detachable programmers includes coding such as, for example, color-coding, corresponding to coding on each of the motorized struts to indicate which motorized strut the detachable programmer should be coupled to. Each of the plurality of detachable programmers is color-coded to one of the plurality of motorized struts.

In any preceding or subsequent example, each of the detachable programmers is arranged and configured to automatically identify which one of the plurality of motorized struts it is connected to in order to ensure proper adjustments.

In any preceding or subsequent example, each of the detachable programmers is arranged and configured as a self-contained, battery-powered cartridge or module.

Examples of the present disclosure provide numerous advantages. For example, by providing self-contained programmers that can be detachably coupled to motorized struts in a spatial frame, the need for any external cables or wires that could snag during use can be eliminated along with the need for incorporating a centralized control unit onto one of the platforms of the spatial frame thereby reducing bulk and safety risk to the patient. In addition, by providing detachable self-contained programmers that can be mechanically and electrically coupled to motorized struts in a spatial frame, a simplified motorized strut can be designed and manufactured. That is, by housing active components of the system in the external detachable programmer, a more compact, easier to produce and less expensive motorized strut is achievable. In addition, the programmer can be attached to the motorized strut in a fracture clinic negating, or at least minimizing, the need for any sterilization. As such, a majority of the control and power circuitry is moved to a separate, external detachable programmer enabling space savings (e.g., space conservation) within the motorized struts and enabling a dumber motorized strut that is easier to manufacture and sterilize.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates a perspective view of a conventional spatial frame including first and second platforms and a plurality of manually adjustable struts coupled thereto;

FIG. 2 illustrates a cross-sectional view of a motorized strut that may be used in a spatial frame such as, for example, within the spatial frame shown in FIG. 1;

FIG. 3A illustrates a perspective view of an example of a spatial frame including a plurality of motorized struts in accordance with one or more features of the present disclosure, each of the motorized struts including a coupler to receive a detachable programmer;

FIG. 3B illustrates an alternate, perspective view of the spatial frame shown in FIG. 3A, each of the plurality of motorized struts including a detachable programmer coupled thereto;

FIG. 4A illustrates a perspective view of the motorized strut shown in FIG. 3A with a portion of the outer body removed;

FIG. 41B illustrates a detailed perspective view of the motorized strut shown in FIG. 4A with a portion of the outer body removed;

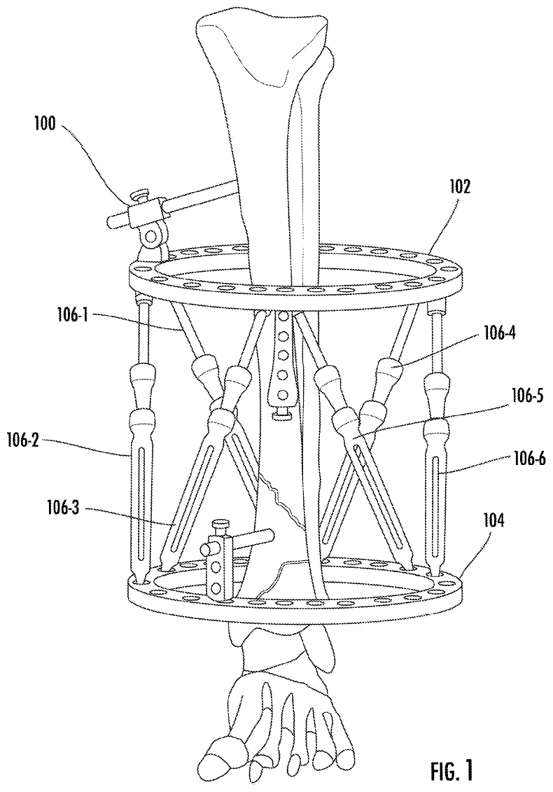

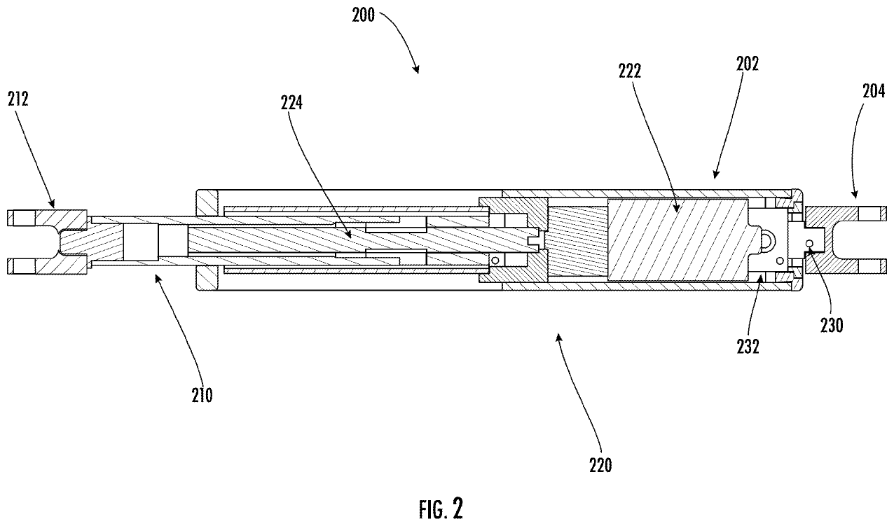

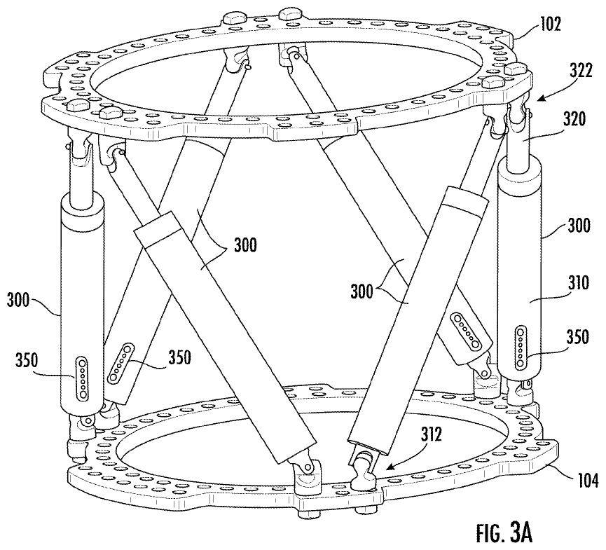

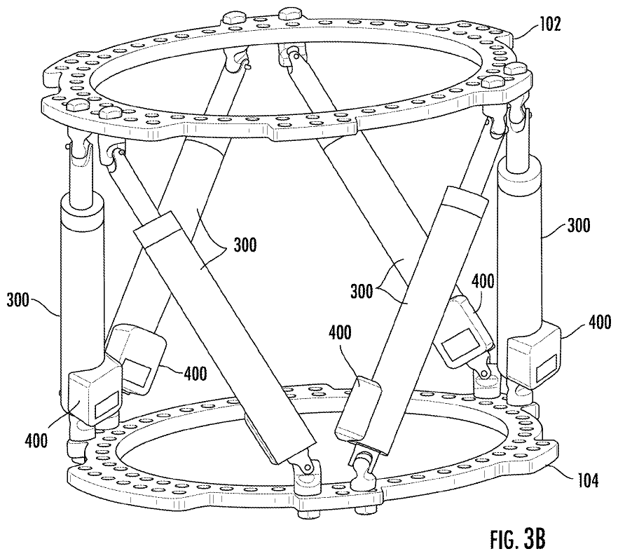

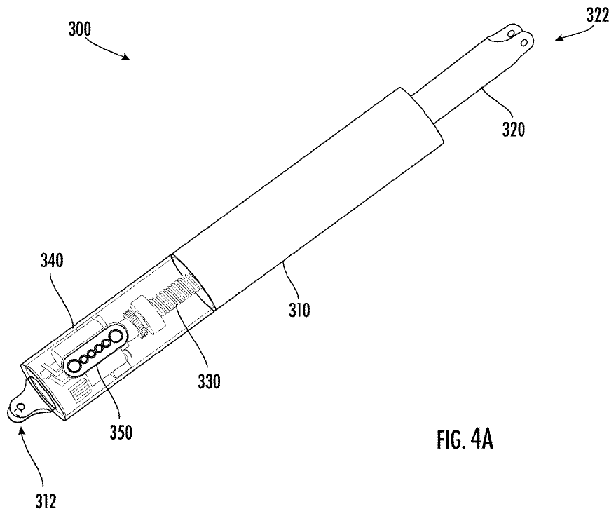

FIG. 5 illustrates a perspective view of the motorized strut and detachable programmer shown in FIG. 3B;

FIG. 6A illustrates a perspective view of an example of a detachable programmer in accordance with one or more features of the present disclosure;

FIG. 6B illustrates an alternate perspective view of the detachable programmer shown in FIG. 6A, the detachable programmer illustrated with the cover removed;

FIG. 6C illustrates a perspective view of an example of a coupler used in the detachable programmer of FIGS. 6A and 6B in accordance with one or more features of the present disclosure;

FIGS. 7A-7C illustrate various perspective views of various alternate examples of a coupling mechanism or couplers that may be used in the motorized struts and detachable programmer in accordance with one or more features of the present disclosure, FIG. 7A illustrates various views of a 4-wire couple configuration, FIG. 7B illustrates various views of a 6-wire couple configuration, and FIG. 7C illustrates various views of a 8-wire couple configuration;

FIG. 8 illustrates various perspective views of an alternate example of a coupling mechanism or couplers that may be used in the motorized struts and detachable programmer in accordance with one or more features of the present disclosure;

FIG. 9 illustrates a perspective view of an example of a digital ecosystem in accordance with one or more features of the present disclosure, FIG. 9 illustrates a detachable programmer wirelessly connected to the cloud via an APP running on a smartphone;

FIGS. 10A-10C illustrate various diagrams of an example of a brushless motor that may be used in the motorized struts in accordance with one or more features of the present disclosure, the brushless motor utilizing an 8-wire configuration;

FIGS. 11A-11C illustrate various diagrams of an example of a stepper motor that may be used in the motorized struts in accordance with one or more features of the present disclosure, the stepper motor utilizing either a 4-wire configuration, a 6-wire configuration, or an 8-wire configuration; and

FIG. 12 illustrates a block diagram of an example of the hardware architecture of the control unit highlighting the actuator (motorized strut with lead screw) and the detachable programmable in accordance with one or more features of the present disclosure.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of a detachable programmer arranged and configured to be coupled to a motorized strut used in an automated and/or motorized spatial frame will now be described more fully herein with reference to the accompanying drawings, in which one or more features of the detachable programmer and/or motorized strut will be shown and described. It should be appreciated that the various features may be used independently of, or in combination, with each other. It will be appreciated that the detachable programmer and/or motorized strut as disclosed herein may be embodied in many different forms and may selectively include one or more concepts, features, or functions described herein. As such, the detachable programmer and/or motorized strut should not be construed as being limited to the specific examples set forth herein. Rather, these examples are provided so that this disclosure will convey certain features of the detachable programmer and/or motorized strut to those skilled in the art.

In accordance with one or more features of the present disclosure, a motorized strut arranged and configured to be used in an automated and/or motorized spatial frame is disclosed. In accordance with one or more features of the present disclosure, the motorized strut is arranged and configured to couple to a detachable programmer. That is, in accordance with one or more features of the present disclosure, a detachable programmer arranged and configured to couple to a motorized strut is also described. In some examples, a plurality of detachable programmers may be provided, one for each motorized strut in the automated and/or motorized spatial frame. Thus arranged, in use, a detachable programmer may be coupled to each of the motorized struts. As will be described in greater detail herein, the detachable programmers may be arranged and configured to control the motorized strut to which it is coupled (e.g., activate the electric motor of the motorized strut according to a treatment plan), supply power to the motorized strut to which it is coupled, and receive and transmit data with an external computing system. Thus arranged, in use, the detachable programmers may be arranged and configured to control and power the motorized struts according to the treatment plan.

In some examples, in accordance with one or more features of the present disclosure and as will be described in greater detail herein, the detachable programmers may be magnetically coupled to the motorized struts. Thus arranged, the detachable programmers can be selectively attached, coupled, docketed, etc. (terms used interchangeably herein without the intent to limit or distinguish) onto each of the motorized struts, either individually or sequentially, and used to actuate the motorized strut according to the treatment plan. As such, the need for any complex, sensitive circuitry housed within the strut may be negated, or at least minimized, thereby easing manufacturability of the motorized struts and/or decreasing the associated costs of the motorized struts. In use, with each of the motorized struts being coupled to a detachable programmer, each of the motorized struts can be programed independently in terms of distraction rate, distraction rhythm, and overall distraction length.

With reference to FIGS. 3A and 3B, an example of a motorized strut 300 in accordance with one or more features of the present disclosure is shown. In use, the motorized strut 300 may be used in a spatial frame in place of conventional manually adjustable struts. For example, the motorized struts 300 may be used in spatial frame 100 in place of the manually adjustable struts 106. In use, the motorized struts 300 facilitate motorized and/or automated adjustments such as, for example, semi-continuous actuation. For example, the motorized struts 300 may enable motorized adjustments to be made autonomously via a companion APP running on, for example, a smartphone, a tablet, or other external computing system. Thus arranged, the spatial frame and/or system architecture may be arranged and configured to automatically adjust the motorized struts 300 according to the prescribed treatment plan (e.g., automatically adjust the plurality of motorized struts 300 without patient intervention). Alternatively, and/or in addition, the spatial frame and/or system architecture may be arranged and configured to require patient and/or caregiver activation to begin the process of automatically adjusting the motorized struts 300 according to the prescribed treatment plan. For example, the spatial frame may be arranged to intermittently auto-adjust the motorized struts 300 at predetermined times according to the treatment plan. Alternatively, the spatial frame may be arranged to intermittently auto-adjust the motorized struts 300 at selected times when convenient and/or when selected by the patient.

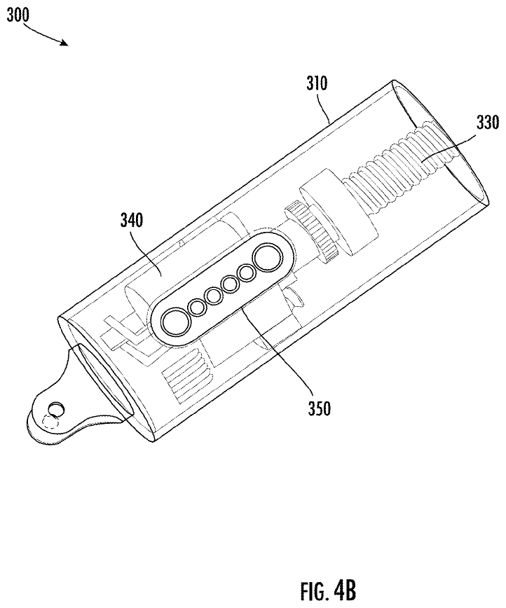

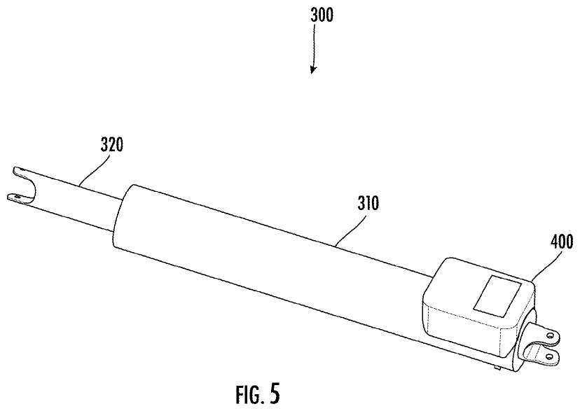

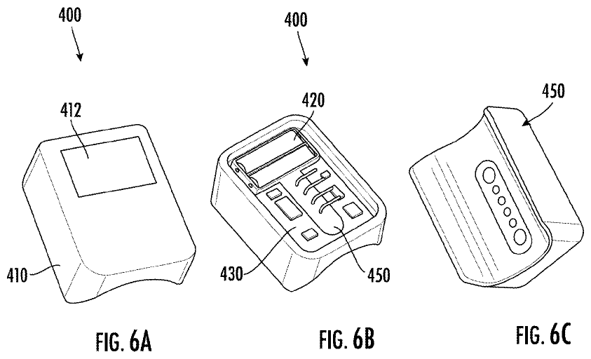

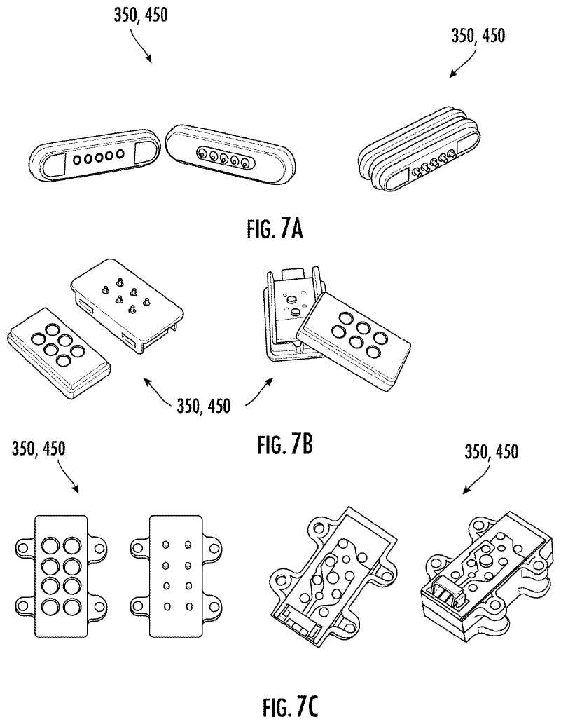

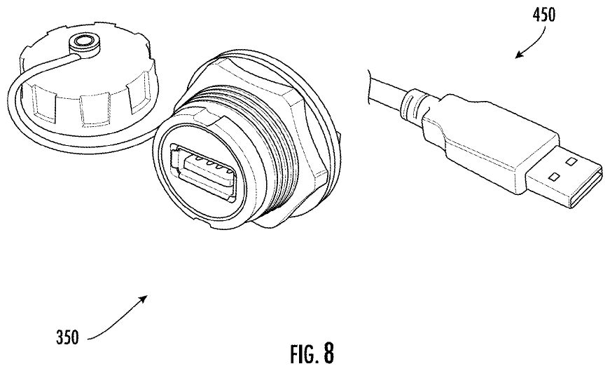

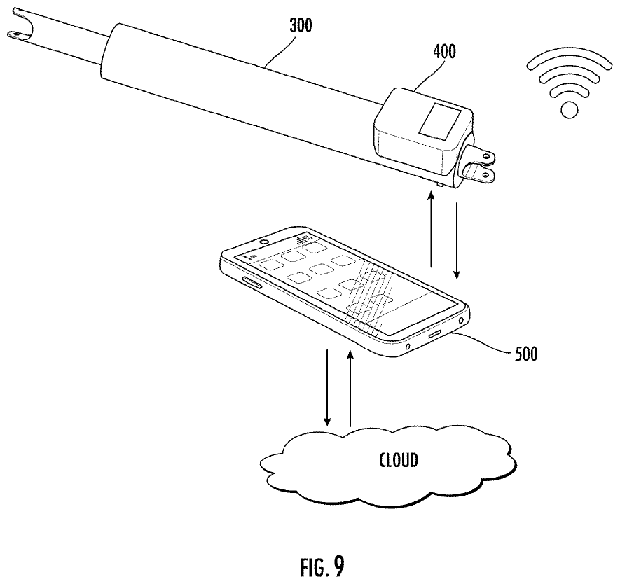

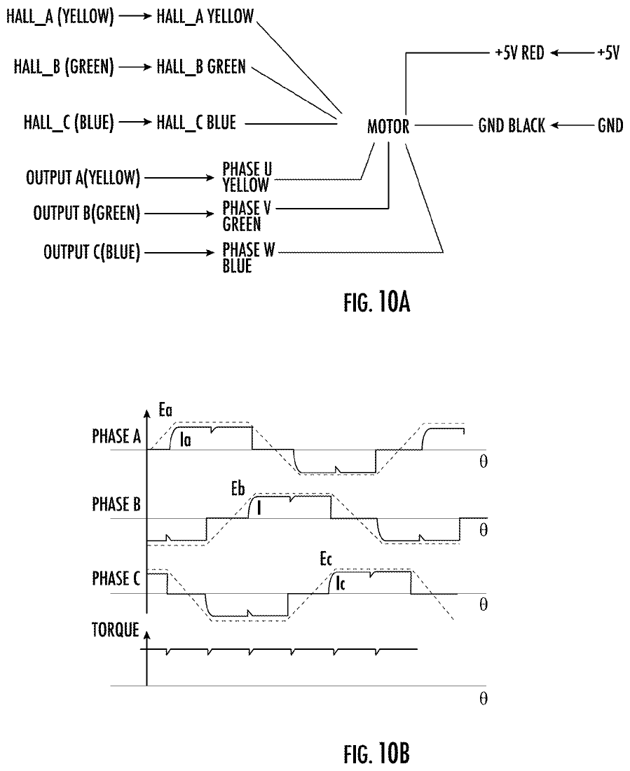

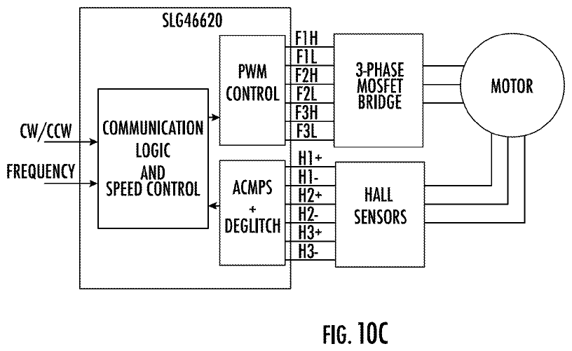

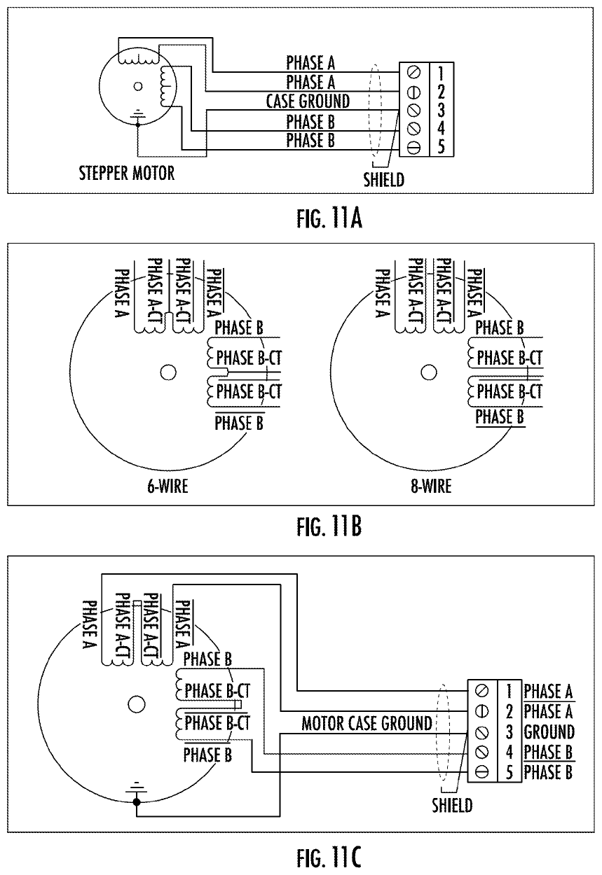

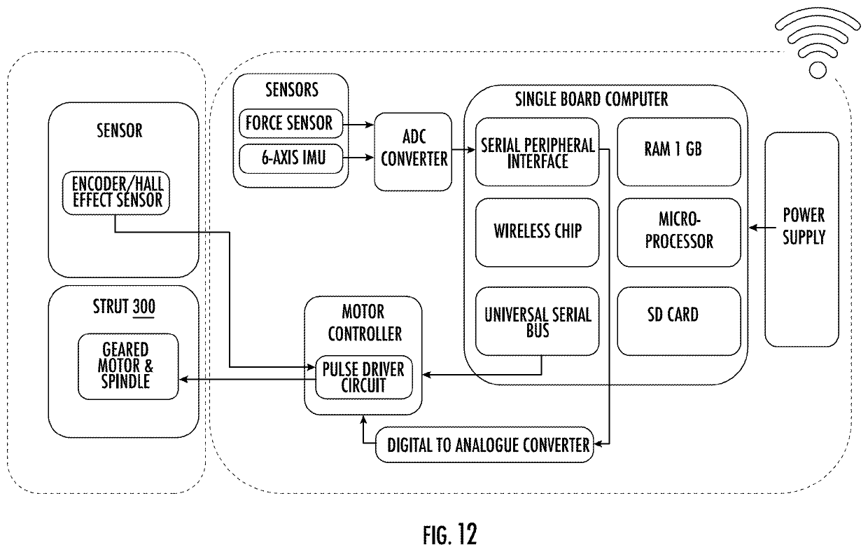

With additional reference to FIGS. 4A, 4B, and 5, in accordance with one or more features of the present disclosure, the motorized struts 300 may include an outer body 310 including a first joint 312 for coupling to a platform such as, for example, platform 104, an inner body 320 including a second joint 322 for coupling to a platform such as, for example, platform 102, an externally threaded lead screw 330 operatively coupling the inner body 320 to the outer body 310 such that rotation of the lead screw 330 moves the inner body 320 relative to the outer body 310 to lengthen or shorten the length of the motorized strut 300 depending on the direction of rotation.

In addition, the motorized strut 300 includes a motor 340 arranged and configured to rotate the lead screw 330, a coupling mechanism or coupler 350 (terms used interchangeably herein without the intent to limit or distinguish) arranged and configured to receive a detachable programmer 400, as will be described in greater detail below, and all necessary components and circuitry so that activation of the motor 340 moves (e.g., rotates) the lead screw 330, and hence adjusts the length of the motorized strut 300. That is, as will be described in greater detail herein, the motorized strut 300 includes a coupler 350 arranged and configured to selectively receive the programmer 400. In some examples, the coupler 350 may be a magnetic coupler such as, for example, a magnetic 4-pin, 6-pin, or 8-pin connector embedded in the external housing of the motorized strut 300. As illustrated, the coupler 350 may be configured to be accessible through the outer body 310 of the motorized strut 300 along a side thereof. As will be appreciated, in use, the motor output wires are soldered to the reverse side of the coupler 350.

As illustrated in FIGS. 3A and 3B, in some examples, a plurality of detachable programmers 400 may be provided, one for each of the motorized struts 300 in the spatial frame. For example, six detachable programmers 400 may be provided, one for each of the six motorized struts 300 in the spatial frame.

With reference to FIGS. 6A-6C, an example of a detachable programmer 400 is illustrated. As illustrated, each detachable programmer 400 includes a housing 410 including a display (e.g., an LED display) 412. In some examples, the housing 410 may be manufactured from a lightweight plastic. In some examples, as will be described herein, the detachable programmer 400 may include all necessary components and/or circuitry needed to control and/or power the motorized struts 300. For example, the detachable programmer 400 may include one or more microcontrollers or microprocessors, one or more wireless communication chips or antennas, one or more power supplies 420 such as, for example, one or more batteries, a charging circuit, a control circuit 430, flash memory for storing data such as, for example, positional data, etc., and any other circuitry or components needed to operate the motorized struts 300 as described herein. In addition, the detachable programmer 400 includes a coupler 450 for mechanically and electrically engaging the motorized strut 300. That is, the detachable programmer 400 includes a coupler 450 arranged and configured to couple (e.g., mechanically and electrically) with the coupler 350 of the motorized strut 300. Thus arranged, in use, each detachable programmer 400 may be selectively coupled to a motorized strut 300 and can be used to power, control, and/or program a motorized strut 300 to enable automated adjustment. In some examples, the detachable programmer 400 is arranged and configured for autonomous bone distraction of the motorized struts 300.

That is, in accordance with one or more features of the present disclosure, the detachable programmer 400 includes a coupler 450 for selectively coupling to a motorized strut 300 (e.g., the detachable programmer 400 includes a coupler 450 arranged and configured to couple (e.g., mechanically and electrically) with the coupler 350 of the motorized strut 300). In addition, the detachable programmer 400 may be arranged and configured to control and power the motorized strut 300 to which it is coupled. That is, in use, each detachable programmer 400 may include any necessary circuitry and power supply to control and power the motorized strut 300 to which it is coupled. Thus arranged, as illustrated, the detachable programmer 400 may be configured as a detachable module or cartridge arranged and configured to be selectively coupled to a motorized strut 300. In use, the detachable programmer 400 can be detached from the motorized strut 300 to, for example, recharge the power supply as needed.

In use, the detachable programmers 400 and the motorized struts 300 may include any suitable coupler now known or hereafter developed for coupling to each other. In some examples, the coupler 350, 450 is arranged and configured to securely couple the detachable programmer 400 to the motorized strut 300 while enabling the detachable programmer 400 to be removed when desired. In addition, in some examples, the couplers 350, 450 enable power and data to be transmitted between the detachable programmer 400 and the motorized strut 300. For example, the detachable programmer 400 can be mechanically fastened to the motorized strut 300. For example, the programmer 400 may be coupled to the strut 300 via one or more fasteners or screws. Alternatively, the programmer 400 may be coupled to the strut 300 via a clamp such as, for example, one or more adjustable band clamps or the like. In a preferred example, the detachable programmer 400 can be magnetically coupled to the motorized strut 300, as will be described in greater detail herein. Thus arranged, in use, the detachable programmer 400 can be arranged as individual battery-powered strut programmers, which can be magnetically attached to each of the motorized struts 300 allowing the motorized struts 300 to be programmed and/or controlled individually. In other examples, the programmer 400 may be coupled to the motorized strut 300 by any suitable connection mechanism now known or hereafter developed such as, for example, an adhesive, cable ties or wrap ties, snap-fit mechanisms, etc.

As previously mentioned, in a preferred example, magnetic coupling or couplers 350, 450 may be provided between the detachable programmer 400 and the motorized strut 300. That is, as best illustrated in FIGS. 3A, 3B, 4A, 4B, 6B, 6C, and 7A-7C, each of the motorized struts 300 and the detachable programmers 400 may include a self-mating magnetic connector or coupler 350, 450. Thus arranged, in order to achieve mechanical and electrical connection between the detachable programmer 400 and the motorized strut 300, magnetic connectors or couplers 350, 450 are provided in each of the motorized struts 300 and the detachable programmers 400, respectively, to provide a magnetic force to both make and maintain a connection therebetween. In use, as best illustrated in FIGS. 7A-7C, the magnetic connectors or couplers 350, 450 may include two mating parts such as, for example, a plug and a socket, having corresponding magnets or magnetic faces that enable a self-aligning connection to be made allowing the connector pins to mate. As generally illustrated in FIGS. 7A-7C, the magnetic connectors or couplers 350, 450 may be in the form of a Pogo interface connector or magnetic pogoes including male pins and female connectors in either 4-pin (FIG. 7A), 6-pin (FIG. 7B), or 8-pin (FIG. 7C) configurations, although this is but a few configurations and other configurations are envisioned. In use, magnetic connectors or couplers 350, 450 allow for easy break-away connections. Thus arranged, in use, electrical coupling between the detachable programmer 400 and the motorized strut 300 can be achieved through a self-mating, male and female magnetic coupler, which relies on a magnetic force to make and maintain a connection during strut adjustment. In use, the detachable programmers 400 may be attached to each motorized strut 300 allowing them to be adjusted individually according to the desired distraction rate, rhythm and distraction length.

However, it should be appreciated that alternate mechanisms for coupling the programmer 400 to the motorized strut 300 are envisioned. For example, the magnetic connectors or couplers 350, 450 may be panel or PCB mount whilst others may be cable mounted, depending on the intended application. For example, with reference to FIG. 8, the programmers 400 may be physically attached to the motorized struts 300. In addition, the programmers 400 may be electrically coupled to the motorized struts 300 using, for example, an IP68 rated waterproof panel connector 350. In some examples, the programmer 400 may be electrically coupled to the motorized strut 300 using, for example, a USB style cable and connector 450. That is, as schematically illustrated, the motorized strut 300 may include a USB panel connector 350 extending from a sidewall thereof. In use, the USB panel connector 350 may be coupled (e.g., soldered) to the motor wires. The programmer 400 may include a USB cable and connector 450 arranged and configured to be received by the USB panel connector 350 of the motorized strut 300.

In some examples, as previously mentioned, each detachable programmer 400 may include a microcontroller or microprocessor arranged and configured to control operation of the motorized strut 300 to which it is coupled including, for example, controlling activation of the motor 340 and/or receiving and/or updating a treatment plan without the need for a separate centralized control unit positioned on or within the spatial frame.

In some examples, as previously mentioned, as best illustrated in FIG. 6B, each detachable programmer 400 may include one or more power supplies 420 such as, for example, one or more batteries, to power itself along with the motorized strut 300 to which it is connected including, for example, the motor 340, the microcontroller, the wireless communication chip, and any associated sensors and/or additional circuitry. In some examples, the detachable programmers 400 are preferably battery-powered. For example, the detachable programmer 400 can be powered using rechargeable batteries such as, for example, rechargeable lithium ion or single cell lithium polymer (“LiPo”) batteries such as, for example, custom-design single cell lithium polymer batteries, with the latter providing more flexibility in terms of shape and capacity. Thus arranged, in use, the detachable programmers 400 may be detached from the motorized strut 300 and docked onto a charging unit each night, or as needed, to allow the batteries to recharge. In some examples, the rechargeable batteries may be encased within the housing of the detachable programmer 400 and cannot be removed by the user. Alternatively, in some examples, the detachable programmer 400 can be powered using non-rechargeable or disposable batteries (e.g., non-rechargeable coin cells or AAA or AA batteries or rechargeable, custom-designed LiPo cells), which may be used and replaced as needed. Alternatively, it is envisioned that wireless charging of the power supply may be utilized.

In some examples, each of the detachable programmers 400 may include, or be operatively associated with, one or more sensors, which may be positioned within the motorized strut, to sense and monitor the functional status of a patient in their natural environment, without the supervision of a doctor or researcher. For example, each of the detachable programmers 400 may include one or more sensors or be in operative communication with one or more sensors positioned within the motorized strut 300 to which it is coupled. For example, a motion sensor such as, for example, an accelerometer, a gyroscope, etc. In use, the motion sensor may be included within the housing of the detachable programmer 400 or within the housing of the motorized strut 300. In use, an accelerometer is an electromechanical device used to measure acceleration forces. Such forces may be static like the continuous force of gravity or may be dynamic to sense movement or vibrations. In use, the gyroscope can be used to measure position, acceleration and rotational motion/angular velocity respectively. In either event, the motion sensor can be programmed to monitor patient activity in terms of total number of steps, total number of sit-to-stand transitions, the ratio of physically active time/rest time, and average walking cadence etc. The accelerometer or gyroscope can also be used to measure the alignment and degree of motion between the bone fragments during the correction phase to check the progression of the prescription plan.

In addition, and/or alternatively, additional sensors may be used. For example, passive strain gauges can be attached to the inner surface of the housing of the motorized strut 300. The strain gauges may be attached in two different planes of the spatial frame to obtain bending strains in the sagittal and frontal planes. These gauges can be oriented perpendicular to the half pins registering bending in the sagittal plane, whereas those oriented parallel to the half pins register bending in the plane perpendicular to the sagittal plane providing the anterior-posterior component of the force in the tibia. These forces can be determined and transmitted to the external computing system and/or displayed on the display 412 of the detachable programmer 400 during the bone healing process to help infer patient-specific treatments or even provide an early warning of non-union.

In some examples, the lead screw may include a magnet, such as a permanent magnet, disposed on a distal end thereof. In these examples, a detector such as, for example, a sensor arranged and configured to produce a sensor signal as a function of a magnetic field, such as a hall sensor, disposed at a distal termination of the lead screw. In some examples, Hall sensors or proximity sensor can be used as sensors to detect magnetic fields and be arranged and configured to produce a signal as a function of a detected magnetic field emitted by the lead screw. In such cases, the displacement signal produce by the detector (sensor) can be correlated to distance d between the lead screw and the detector (sensor). In some other examples, the displacement signal can be generated as a function of a sensed electric field, for example when capacitive displacement sensing is used as displacement sensor.

In addition, and/or alternatively, the detachable programmer 400 may include or be operatively associated with, one or more positional sensors, which may be positioned within the motorized strut, to monitor the length of the motorized strut 300, load sensors for providing biomechanical feedback during bone healing, acoustic emission or vibration sensors for fault level detection in the gear train, etc.

In some examples, as previously mentioned, each detachable programmer 400 may include a wireless communication chip or antenna arranged and configured to communicate with an external computing system to, for example, exchange data with the external computing system via, for example, an APP running on, for example, a mobile phone 500. For example, the data may relate to strut position, exchange data relating to and updating the prescribed treatment plan, etc. That is, each detachable programmer 400 may be arranged and configured to receive and transmit data to, for example, a mobile phone or remote computing system relating to the treatment plan. Data may include, for example, distraction length, lengthening direction, rate and rhythm of distraction, total amount of distraction, lengthening schedule, number of turns of the motor/gear assembly, date and time, or health or diagnostic information such as, for example, the health of the strut (battery life/voltage, and error events relating to the motor, over current, over voltage, temperature, etc. In some examples, each detachable programmer 400 can receive and transmit data from the motorized strut 300 to a mobile app relating to (a) patient compliance, (b) healing status (via the force exerted by the actuator, which can be correlated with the current consumed by the motor), (c) treatment plan, i.e distraction length, lengthening direction, rate and rhythm of distraction, total amount of distraction, lengthening schedule, number of turns of the motor/gear assembly, date and time and (d) the health of the strut (battery life/voltage, and error events relating to the motor (over current, over voltage, temperature). In addition, and/or alternatively, this information can also be displayed on the display 412 of the detachable programmer 400.

In some examples, in use, six individual detachable programmers 400 can be provided, one for each motorized strut 300, although it is envisioned that less detachable programmers 400 can be provided and/or that individual detachable programmers 400 can be used to detachably couple and/or control more than one motorized strut 300. For example, it is envisioned that a single detachable programmer could be used by sequentially, by attaching the detachable programmer to a first strut then a second strut, and so-on. In one preferred example, a detachable programmer would be connected to each of the plurality of motorized struts and would remain attached until charging was needed, or until completion of the treatment period. Thus arranged, continuous autonomous control of the motorized struts could be achieved.

In some examples, the detachable programmers 400 are arranged and configured to identify which motorized strut 300 it is or should be coupled to. For example, the detachable programmer 400 may be coded such as, for example, color-coded, to identify what motorized strut 300 the detachable programmer 400 should be coupled to. For example, the detachable programmer 400, along with the corresponding motorized strut 300, may be color-coded red, orange, yellow, green, blue, and purple to assist with identifying which motorized strut 300 to couple to.

Alternatively, the detachable programmer 400 can be arranged and configured to automatically identify which motorized strut 300 of the spatial frame it is connected to in order to ensure proper adjustments according to the treatment plan are being made. For example, the detachable programmer 400 is able to identify which motorized strut 300 it is attached to in order to adjust each strut correctly. In use, identification of which motorized strut 300 the detachable programmer 400 is coupled to can be performed by any mechanism or method now known or hereafter developed. Identification can be accomplished wirelessly or through the physical coupling (e.g., magnetic couplers). For example, a color sensor could be included in the detachable programmer 400, which could be used to scan color coded parts on each of the motorized struts 300. In some examples, a specific motorized strut 300 to which the detachable programmer 400 is being coupled to can be identified by scanning a QR code label on the motorized strut 300, which may be activated by a user scanning an image (the code) with a camera APP. Alternatively, tags such as, for example, NFC tags or Bluetooth connections having a unique code, called UID (Unique ID), can be integrated within the housing of each motorized strut 300 and be read by most software systems, including iPhone and Android 3^{rd} party APPs. In another example, a 2D barcode can be located on the external surface of the housing of the motorized strut 300 and be read by a nearby scanner or the motorized strut 300 can be detected electronically by a MAC address, which may be, for example, a 48-bit hexadecimal address that can be used as a unique hardware identification number.

In accordance with one or more features of the present disclosure, by coupling individual detachable programmers 400 to each of the motorized struts 300, self-contained units or devices are provided. For example, the detachable programmer 400 may be configured as wireless, self-powered, and independently operated devices (e.g., the detachable programmer 400 are arranged and configured as a self-contained unit including all of the necessary components and circuitry to control the coupled motorized strut 300 according to the prescribed treatment plan). Thus arranged, the motorized struts 300 with the detachable programmer 400 coupled thereto eliminate the need for any external cables or wires that could snag during use and eliminate the need for incorporating a centralized control unit onto one of the platforms of the spatial frame thereby reducing bulk and safety risk to the patient (e.g., self-containment of the control circuitry, wireless communication chip, and power source within detachable programmer 400 negate the need for cables and a centralized control unit positioned elsewhere on the spatial frame along with any needed cables or wires).

As previously mentioned, in some examples, when arranged in a spatial frame, the motorized struts 300 with the detachable programmer 400 coupled thereto may be arranged and configured to wirelessly exchange data, instructions, etc. with an external computing system such as, for example, a smartphone 500, a tablet, a computer, etc. running a companion APP. However, it is envisioned that the motorized struts 300 may exchange data with an external computing system by any now known or hereafter developed system. For example, each of the motorized struts 300 may include a communication interface to exchange data over a wired connection.

As previously mentioned, each of the motorized struts 300 includes a geared electric motor 340 and a lead screw 330. In some examples, the motor 340 may be positioned in-line with the lead screw 330 (e.g., longitudinal central axis of the lead screw 330 is parallel and aligned with the longitudinal central axis of the motor 340), although the motor 340 may be positioned offset or off-axis from the lead screw 330. In use, the motor 340 can be any suitable motor now known or hereafter developed including, for example, an AC induction motor, a brushless DC motor, a brushed DC motor, a stepper motor, a servo motor, etc. In the case of a brushless motor, the motorized strut 300 may also include one or multiple Hall Effect sensors for detecting the rotor position from the magnetic field, which are mounted either to the stator or to the rotor.

In use, to enable electrical contact with the detachable programmer, the motor wires may be re-routed through the sidewall of the housing of the motorized strut 300. In some examples, the motor wires may be soldered onto a suitable magnetic coupler 350. In use, selection of the couplers 350, 450 may be dependent on the type of motor being utilized (e.g., the Pogo interface connector or magnetic pogo pin connector and pin layout may be dependent on the type of motor being utilized). For example, brushed DC motors (BDC), utilize three wires (i.e., two wires or pins to power the DC motor (+VCC and Ground) and one wire or pin to support the absolute linear encoder). Meanwhile, as schematically illustrated in FIGS. 10A-10C, a brushless DC motor (BLDC) utilizes a more complex driving circuitry, which controls the AC current required to drive them. As such, BLDC motors utilize eight wires or pins (e.g., three wires or pins for power for the phases of the motor, two wires or pins for power to the hall sensors, and three wires or pins for the hall sensors). FIGS. 10A-10C schematically illustrate a BLDC motor 8-wire configuration highlighting the main block diagram for the 3-Phase brushless DC motor circuit. BLDCs use three wires, which are driven by the ESC with a phase-shifted AC waveform. Each wire's waveform is shifted by 120 degrees from the other two. Phase A, B and C refer to yellow, green and blue wires respectively.

In some examples, the motor 340 may be a stepper motor. For example, with reference to FIGS. 11A-11C, an example of a stepper motor is illustrated. In use, a stepper motor can be provided with an assortment of wire configurations including a 4-wire configuration (FIG. 11A), a 6-wire configuration (FIG. 11B) and an 8-wire configuration (FIG. 11B). A basic 4-wire stepper motor is shown in FIG. 11A and requires connecting the A and A′ leads to the corresponding phase outputs on the motor drive. A stepper motor equipped with four wires means that it can only be used with a bipolar driver. A 6-wire stepper motor is similar to a 4-wire configuration with the added feature of a common tap placed between either end of each phase as shown FIG. 11B. Stepper motors with these center taps are referred to as unipolar motors. This wiring configuration is best suited for applications requiring high torque at relatively low speeds such as motorized external fixators. The 8-Wire configuration, shown in FIG. 11B, allows for multiple wiring configurations depending on whether the motor's speed or torque is more important. An 8-wire stepper motor can be connected with the windings in either series or parallel.

With reference to FIG. 12, an example of a block diagram of the hardware architecture of a detachable programmer is illustrated. As generally illustrated, the power and control unit highlighting the electrical connection between the actuator (motorized strut with lead screw) and the programmable motor controller. As illustrated, in some examples, the detachable programmer includes a force sensor and a 6-axis IMU (accelerometer and gyroscope), single board computer, power supply, motor controller software and firmware, micro-processor, flash memory, Digital-to-Analog and Analog to Digital Converter, and a wireless chip for transferring strut status/information to a mobile app and web-based interface.

In accordance with one or more features of the present disclosure, the motorized struts 300 may also ease the process of sterilization since the detachable programmers 400 can be coupled to the motorized struts 300 in clinic (e.g., by moving the control circuit and power supply to the external detachable programmer, the motorized struts can be sterilized using, for example, gamma radiation without the concern of damaging the circuits or power supply).

In some examples, the detachable programmer may be 20 mm×30 mm×15 mm (width×length×height).

In some examples, the motorized struts 300 may be water-proofed to facilitate the patient, for example, taking a shower or bath. Alternatively, it is envisioned that the spatial frame may be covered by, for example, a bag during a shower thus alleviating the necessity for water-proofing each of the motorized struts 300.

In some examples, the motorized struts 300 may include an IP-68 rated housing manufactured from any suitable material including, for example, a metal or metal alloy, a polymer, a light-weight material such as PEEK, nylon, aluminum, etc. In addition, the housing may be manufactured via any now known or hereafter developed technique such as, for example, injection molding, additive manufacturing, etc.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A motorized spatial frame comprising:
a first platform;
a second platform;
a plurality of motorized struts coupled to the first and second platforms, each of the plurality of motorized struts including an outer body, a lead screw, and a motor operatively coupled to the lead screw for adjusting a position of the lead screw relative to the outer body to adjust a position of the first platform relative to the second platform, each of the plurality of motorized struts further including a coupler; and
one or more detachable programmers including a coupler arranged and configured to selectively engage the coupler of the motorized strut so that the one or more detachable programmers can be selectively attached and detached from the plurality of motorized struts;
wherein each of the one or more detachable programmers is configured to supply power and to control activation of the motorized strut to which the detachable programmer is attached to, the detachable programmer configured to actuate the motorized strut according to a treatment plan.

2. The motorized spatial frame according to claim 1, wherein the one or more detachable programmers include a plurality of detachable programmers, one for each of the plurality of motorized struts.

3. The motorized spatial frame according to claim 1, wherein the coupler on the detachable programmer and the coupler on the motorized are arranged and configured to mechanically and electrically couple the detachable programmer to the motorized strut.

4. The motorized spatial frame according to claim 3, wherein the coupler on the detachable programmer is arranged and configured to magnetically couple to the coupler on the motorized strut so that the detachable programmer is magnetically attached to the motorized strut.

5. The motorized spatial frame according to claim 4, wherein the magnetic couplers on the detachable programmer and the motorized strut include a Pogo interface connector including male pins and female connectors in one of a 4-pin, 6-pin, or 8-pin configuration.

6. The motorized spatial frame according to claim 1, wherein each of the detachable programmers include one or more microcontrollers or microprocessors arranged and configured to control the detachable programmer and the motorized strut to which it is coupled.

7. The motorized spatial frame according to claim 6, wherein the one or more microcontrollers or microprocessors are arranged and configured to control operation of the motorized strut including controlling activation of the motor and receiving and updating a treatment plan associated with the motorized spatial frame.

8. The motorized spatial frame according to claim 1, wherein each of the detachable programmers include one or more power supplies arranged and configured to power the detachable programmer and to supply power to the motorized strut to which it is coupled.

9. The motorized spatial frame according to claim 1, wherein each of the detachable programmers include a wireless communication chip arranged and configured to wirelessly communicate with an external computing system to exchange data relating to a treatment plan, to exchange data relating to a status of the motorized strut, to exchange data relating to a health or activity of the patient, or a combination thereof.

10. The motorized spatial frame according to claim 1, wherein each of the detachable programmers is arranged and configured to identify which one of the plurality of motorized struts it is to be coupled to.

11. The motorized spatial frame according to claim 10, wherein each of the detachable programmers includes color-coding corresponding to color-coding on each of the motorized struts to indicate which motorized strut the detachable programmer is to be coupled to.

12. The motorized spatial frame according to claim 10, wherein each of the detachable programmers is arranged and configured to automatically identify which one of the plurality of motorized struts it is connected to in order to ensure proper adjustments.

13. The motorized spatial frame according to claim 12, wherein each of the detachable programmers is arranged and configured to one of scan a QR code label on the motorized strut, read a tag containing a unique ID code on the motorized strut, or read a two-dimensional barcode on the motorized strut.

14. A motorized spatial frame comprising:
a first platform;
a second platform;
a plurality of motorized struts coupled to the first and second platforms, each of the plurality of motorized struts including an outer body, a lead screw, and a motor operatively coupled to the lead screw for adjusting a position of the lead screw relative to the outer body to adjust a position of the first platform relative to the second platform, each of the plurality of motorized struts further including a coupler; and
a plurality of detachable programmers, one for each of the plurality of motorized struts, each of the plurality of detachable programmers including:
a microprocessor or microcontroller arranged and configured to control operation of the motorized strut to which it is coupled,
a power supply arranged and configured to supply power to the motorized strut to which it is coupled,
a wireless communication chip arranged and configured to transmit and receive data with the motorized strut to which it is coupled and with an external computing system; and
a coupler arranged and configured to selectively engage the coupler of one of the plurality of the motorized struts so that the detachable programmer is selectively attached and detached from one of the plurality of motorized struts.
